(19) United States
(12) Patent Application Publication
(10) Pub. No.: US 2008/0058691 A1
(43) Pub. Date: Mar. 6, 2008

(54) BULLET PLUG GUNSHOT WOUND FIRST AID, OPEN OPERATION DEVICE

(76) Inventor: Bradford Tyler Sorensen, Manhattan Beach, CA (US)

Correspondence Address: BRADFORD T. SORENSEN, 515 13TH STREET, MANHATTAN BEACH, CA, 90266, US

(21) Appl. No.: 11/469,040

(22) Filed: Aug. 31, 2006

(51) Int. Cl. A61F 13/00

(52) U.S. Cl. 602/48

(57) ABSTRACT

A bullet plug gunshot wound first aid device (BulletPLUG) that can be scaled in size to insert into a bullet wound, transform from a rolled cylinder of durable flexible clear material into an expanding tunnel of clear unrolled material to open the hole from a bullet wound to expose the bullet's location at the end of the tunnel and provide a clear, light illuminated, open tunnel to facilitate removal of the bullet, while a local coagulant coating on the outside surface of the tunnel stops the bleeding of the bullet wound into the bullet hole, while a powerful local anesthetic reduces the pain of the wound. The current invention also provides an Open Operation (OpenO) device that spreads open an incision to aid in a surgical operation by providing a clear, light illuminated, open tunnel to facilitate sub-dermal access, while a local coagulant coating on the outside surface of the tunnel reduces the bleeding of the incision, while a local anesthetic reduces the pain of the incision.

### BACKGROUND AND SUMMARY OF THE INVENTION

The current invention provides first aid for a gunshot bullet wound.

Specifically, the current invention provides a device that plugs a bullet hole (BulletPLUG) by creating a clear expanded tunnel with which to open the cylindrical hole from a bullet wound to expose the bullet's location and provide a clear, light illuminated, open tunnel to facilitate removal of the bullet, while a local coagulant coating on the outside surface of the tunnel reduces the bleeding of the bullet wound into the hole, while a local anesthetic reduces the pain of the wound. Once the bullet is removed, a plug is inserted within the open tunnel to stop bleeding as well as stop air from going into the bullet hole. The current invention also provides an open operation (OpenO) device that spreads open an incision to aid in a surgical operation by providing a clear, light illuminated, open tunnel to facilitate sub-dermal access, while a local coagulant coating on the outside surface of the tunnel reduces the bleeding of the incision, while a local anesthetic reduces the pain of the incision.

Specifically, the current invention provides a gunshot bullet wound first aid device (BulletPLUG) that consists of a spiral roll of flexible, durable, clear, sheet material that is coated with a local anesthetic and a blood coagulant and is inserted into the bullet wound and is unrolled by a drive mechanism to form a clear cylindrical tunnel which opens up to expose and give easy access to the bullet at the end of the tunnel and displays the lining of the bullet wound through the clear walls of the tunnel to the doctor during an exam or to the gunshot victim or to the person giving first aid. The current invention allows 360 degree viewing of the interior of the gunshot wound for improved diagnosis and direct, open, cylindrical, access to the bullet deep inside the wound for removal. As the clear cylindrical tunnel expands within the gunshot wound, the interior diameter grows slightly larger than the outer diameter, which naturally holds the truncated conical shaped tunnel inside the body at the opening of the wound. The drive mechanism has the multi-functions of providing controlled opening and closing of the clear tunnel, providing a ratchet mechanism that locks the tunnel at any desired diameter and provides an automatic enlargement and reduction of the tunnel maintaining a slightly larger interior diameter than exterior diameter truncated conical shape for conical tunnel retention in the wound.

Historically, first aid of gunshot wounds has involved applying pressure to the bullet wound to reduce the bleeding, or the wound is stuffed full of cotton or cloth to slow down the loss of blood until the gunshot victim can be rushed to a hospital for an emergency operation.

The current invention is the first portable, coagulant, anesthetic, gunshot wound first aid device.

Upon receiving a gunshot wound, the victim (if conscious and able) can easily open the special sterile wrapper, insert the first aid device into the wound hole, open the tunnel diameter of the device and automatically expose the wound to the anesthetic coating to reduce the pain, automatically expose the wound to blood coagulant coating to reduce the bleeding and turn on the light to illuminate and reveal the location of the bullet at the bottom of the tunnel for removal. The bullet plug gunshot wound first aid device kit also includes a liquid inside the sterile wrapper that is an anesthetic to reduce pain and a coagulant to reduce bleeding. The anesthetic/coagulant liquid can be applied to the surface of the wound to reduce pain and/or squeezed down the tunnel to reduce the bleeding at the end of the tunnel before or after the bullet is removed. If the gunshot victim is unconscious or unable to open the sterile wrapper or insert the first aid device himself, a doctor or other person may be available to administer the bullet plug gunshot wound first aid device for the victim following the symbolic instructions printed on the sterile wrapper.

In some cases, a bullet wound extends all the way through the body and exits the body leaving an entry hole and an exit hole. The entry hole is typically smaller than the exit hole. The current invention creates a conical shaped tunnel, which automatically seals both the entry hole and the exit hole and all punctured tissue in between. The cone shape is coated with blood coagulant and anesthetic to reduce the bleeding and the pain of both the entry hole and the exit hole. The bullet plug product will come packaged in a variety of sizes to accommodate small caliber bullets, large caliber bullets, shotgun pellets, shallow wounds, deep wounds, and through-hole wounds. In the case of shotgun pellet wounds, multiple small diameter plugs are provided to plug the large number of individual pellet holes caused by a shotgun blast.

The current invention may be enhanced with the use of a cylindrical balloon concentric with the center of the spiral clear material with an internal pressurized CO2 capsule and a button. The button moves a pointed item that can pierce the capsule, causing the expansion of the cylindrical balloon inside the spiral clear material, which causes an instant filling of the gunshot wound to form a bullet plug.

The current invention is also a sterile, surgical incision-opening device for open Operations (OpenO).

This device can be adapted to examine all bodily orifices, punctures, lacerations, incisions or wounds.

This device can be adapted to become a retractor to hold tissue open during a sub dermal operation.

This device can be adapted to become a substantially circular expanding or retracting ring or tunnel that holds tissue open during an operation.

This device can also be adapted to become a flexible, circular expanding or retracting ring or tunnel that controls bleeding and pain during an operation.

In addition to the anesthetic and blood coagulant, an anti-biotic or soap coating can be added to combat the infection and germ problems and aid the healing process.

### PREVIOUS ART

In the past, no patents have issued relating to bullet wound first aid devices. Several previous art devices relate in a peripheral way to the current invention, but do not address the novel concept of a bullet wound first aid device. Such peripheral previous art devices include; U.S. Pat. No. 5,263,922 issued on Nov. 23, 1993, to Sova, which teaches a valved bandage for treating a collapsed lung caused by a puncture wound in the chest to allow the exit of air and fluids and to stop the entry of air into the lining of the lungs which causes the collapse of the lungs. This previous art deals with a one-way surgical valve for the treatment of collapsed lungs. The current invention is a novel way of providing first aid to gunshot wounds anywhere in the body while providing blood coagulation to stop bleeding and anesthetic to stop pain while providing a tunnel with which to remove the bullet.

U.S. Pat. No. 6,334,870, issued on Jan. 1, 2002, to Timothy Ehr, teaches an expandable spiral stent that includes uncoiling spiral members that form a generally tubular body that keeps arteries open by spiral spring pressure to prevent heart disease. The current invention provides a completely different and novel way of using an expandable spiral roll of clear material coated with a coagulant and an anesthetic to form an open tunnel to remove a bullet from a gunshot wound while providing control of bleeding and pain.

In the past, previous art relating to devices that open incisions during surgical operations include devices known as retractors. Several previous art surgical retractor devices relate in a peripheral way to the current invention, but do not address the novel concept of an open operation device using a spiral of material coated with blood coagulant and anesthetic that expands to create an open tunnel to access the interior of the body. One such related previous art device includes, U.S. Pat. No. 6,264,605 to Scirica on Jul. 24, 2001, which shows a typical surgical retractor with a support frame and adjustable tissue retraction clamps and grips. Such previous retractors simply spread open incisions or wounds for sub dermal access without the accommodation for blood coagulation, pain reduction or antibiotic healing functions offered by the current invention. In addition, the current invention places even pressure around the circular opening instead of point pressures caused by retractor clamps.

The present invention bullet plug comes packaged in sterile combination coagulant, anesthetic and lubrication. This healing lubrication aids the insertion and expansion of the bullet plug first aid device to form a cylindrical or conical seal that stops bleeding and pain.

The present open operation invention provides innovative improvements over the previous art by introducing a more effective and functional device and method for opening, examining and performing sub-dermal operations with pain reduction and control over bleeding.

### OBJECTS OF THE INVENTION

The first object of the present invention is to provide first aid for a gunshot bullet wound.

The second object of the present invention is to provide a gunshot bullet wound first aid device that creates a clear expanded tunnel with which to open the cylindrical hole from a bullet wound to expose the bullet's location.

The third object of the present invention is to provide a gunshot bullet wound first aid device that has a clear, light illuminated, open tunnel to facilitate the removal of the bullet.

The fourth object of the present invention is to provide a gunshot bullet wound first aid device with a local coagulant coating on the outside surface of the tunnel that reduces the bleeding of the bullet wound into the hole.

The fifth object of the present invention is to provide a gunshot bullet wound first aid device with a local anesthetic on the outside surface of the tunnel that reduces the pain of the wound.

The sixth object of the present invention is to provide a gunshot bullet wound first aid device with an anti-biotic or soap on the outside surface of the tunnel to reduce the infection and germ problems associated with a gunshot wound.

The seventh object of the present invention is to provide a gunshot bullet wound first aid device with a rolled up, spiral, tunnel that is inserted into the bullet wound and unrolled by a drive mechanism to form a clear cylindrical tunnel.

The eighth object of the present invention is to provide a gunshot bullet wound first aid device with a rolled up tunnel that opens up to expose and give easy access to the bullet at the end of the tunnel.

The ninth object of the present invention is to provide a gunshot bullet wound first aid device with a transparent tunnel that displays the lining of the bullet wound through the clear walls of the tunnel to the doctor during an exam or to the gunshot victim or to the person giving first aid.

The tenth object of the present invention is to provide a gunshot bullet wound first aid device with a transparent tunnel that allows 360 degree viewing of the interior of the gunshot wound for improved diagnosis.

The eleventh object of the present invention is to provide a gunshot bullet wound first aid device with a transparent tunnel that allows direct viewing of the interior of the gunshot wound for direct, open, cylindrical, access to the bullet deep inside the wound for removal.

The twelfth object of the present invention is to provide a gunshot bullet wound first aid device with an interior diameter that grows slightly larger than the outer diameter, which naturally holds the truncated conical shaped tunnel inside the body at the opening of the wound.

The thirteenth object of the present invention is to provide a gunshot bullet wound first aid device that provides controlled opening and dosing of the clear tunnel.

The fourteenth object of the present invention is to provide a gunshot bullet wound first aid device providing a ratchet mechanism that locks the tunnel size at any desired diameter.

The fifteenth object of the present invention is to provide a gunshot bullet wound first aid device with a vacuum fluid removal and suction function.

The sixteenth object of the present invention is to provide a gunshot bullet wound first aid device that consists of a spiral roll of flexible, durable, clear, sheet material with a concentric balloon containing a pressurized CO2 capsule and a button provided with a sharp item for piercing the capsule for instantaneously expanding the balloon which unrolls the spiral cone inside the wound and applies coagulant and anesthetic while sealing the bullet hole.

The seventeenth object of the present invention is to provide gunshot bullet wound first aid device with an automatic enlargement and reduction of the tunnel maintaining a slightly larger interior diameter than exterior diameter truncated conical shape for conical tunnel retention in the wound.

The eighteenth object of the present invention is to provide a gunshot bullet wound first aid device that conforms to the normal configuration of a bullet wound wherein the hole is small at the surface and expands in diameter as it enters the body using an interior diameter that grows slightly larger than the outer diameter which naturally holds the truncated conical shaped tunnel inside the body at the opening of the wound.

The nineteenth object of the present invention is to provide an alternative to stuffing the bullet wound full of cotton or cloth to slow down the loss of blood until the gunshot victim can be rushed to a hospital for an emergency operation.

The twentieth object of the present invention is to provide an alternative to applying pressure to the bullet wound to reduce the bleeding before emergency help is obtained.

The twenty-first object of the present invention is to provide a gunshot bullet wound first aid device that is small and portable.

The twenty-second object of the present invention is to provide a gunshot bullet wound first aid device that is individually wrapped in a sterilized convenient package.

The twenty-third object of the present invention is to provide a gunshot bullet wound first aid device that includes clear symbolic instructions printed on the sterile wrapper that provides any person with the knowledge to administer the bullet plug gunshot wound first aid device.

The twenty-fourth object of the present invention is to provide a device that spreads open an incision to aid in a surgical operation by providing a clear, light illuminated, open tunnel to facilitate sub-dermal access, while a local coagulant coating on the outside surface of the tunnel reduces the bleeding of the incision, while a local anesthetic reduces the pain of the incision while an anti-biotic or soap kills germs.

### PREFERRED EMBODIMENT COMPONENT LIST

10 bullet plug device
11 typical bullet wound victim
12 bullet wound hole
13 bullet plug left handle
14 bullet plug right handle
15 bullet plug rolled spiral cylinder
16 typical gun barrel
17 explosive propellant burst
18 bullet acceleration path
19 typical section of a body part
20 bullet impact point
21 bullet entering body part
22 bullet impacting bone
23 bullet
24 bleeding bullet wound hole
25 bullet plug entry point
26 bullet plug unrolling spiral cone
27 coagulant coating
28 anesthetic coating
29 bullet wound hole touching coagulant
30 bullet wound hole touching anesthetic
31 expanding unrolled spiral cone
32 compressed wound hole wall
33 fully expanded spiral cone
34 plunger
35 plunger expansion screw
36 plunger stick
37 wound seal area
38 bullet at the end of the wound
40 typical scalpel
41 inner transparent walls of spiral cone
42 typical incision
43 expansion ratchet handle
44 light
50 Open-O Device

### BRIEF DESCRIPTION OF THE DRAWINGS

FIG. 1 shows a perspective view of an embodiment of the present invention bullet plug gunshot wound first aid device in use by a victim of a gunshot wound.

FIG. 2 is a section view of a typical gun barrel firing a bullet into a cross section of a typical human arm with the bullet piercing the skin and striking the bone of the victim.

FIG. 3 is a section view of an embodiment of the present invention bullet plug in a rolled configuration being inserted into the bleeding bullet hole of the victim.

FIG. 4 is a section view of an embodiment of the present invention bullet plug being unrolled by the drive mechanism and expanding in diameter to fill bleeding bullet hole of the victim while causing desired coagulant and anesthetic contact and pressure to shut off the bleeding and reduce the pain of the wound.

FIG. 5 is a section view of an embodiment of the present invention bullet plug in a fully unrolled configuration having the open tunnel plugged by the expandable plunger stick.

FIG. 6 is a perspective view of the current invention Open-O (Open Operation Device) about to be inserted into a typical incision cut by a typical scalpel.

FIG. 7 is a perspective view of the rolled spiral cylinder inserted into the typical incision.

FIG. 8 is a perspective view of the expanding unrolled spiral cone starting to enlarge the typical incision.

FIG. 9 is a perspective view of the fully expanded spiral cone as it exposes the sub dermal organs of the body with in the circle of the spiral cone

### DETAILED DESCRIPTION OF THE EXEMPLARY EMBODIMENT(S)

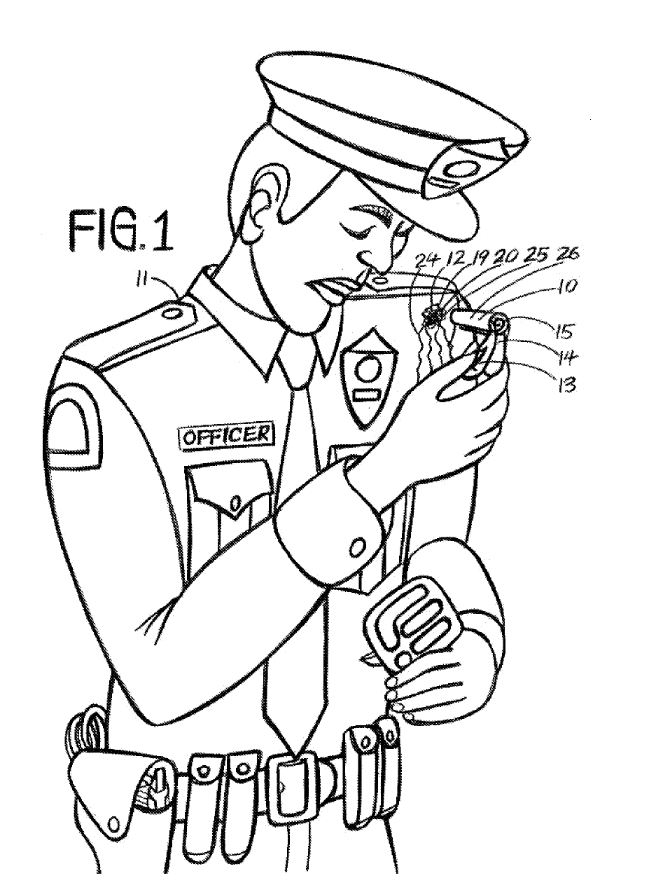

FIG. 1 shows the present invention 10 bullet plug device being utilized by a 11 typical bullet wound victim to provide first aid for a gunshot wound by inserting the 10 bullet plug device into the 12 bullet wound hole holding the 13 bullet plug left handle and the 14 bullet plug right handle which guides the 15 bullet plug rolled spiral cylinder into the 12 bullet wound hole in the 20 bullet impact point on a 19 typical section of a body part. The 26 bullet plug unrolling spiral cone portion of the 10 bullet plug device is inserted into the 24 bleeding bullet wound hole at the 25 bullet plug entry point.

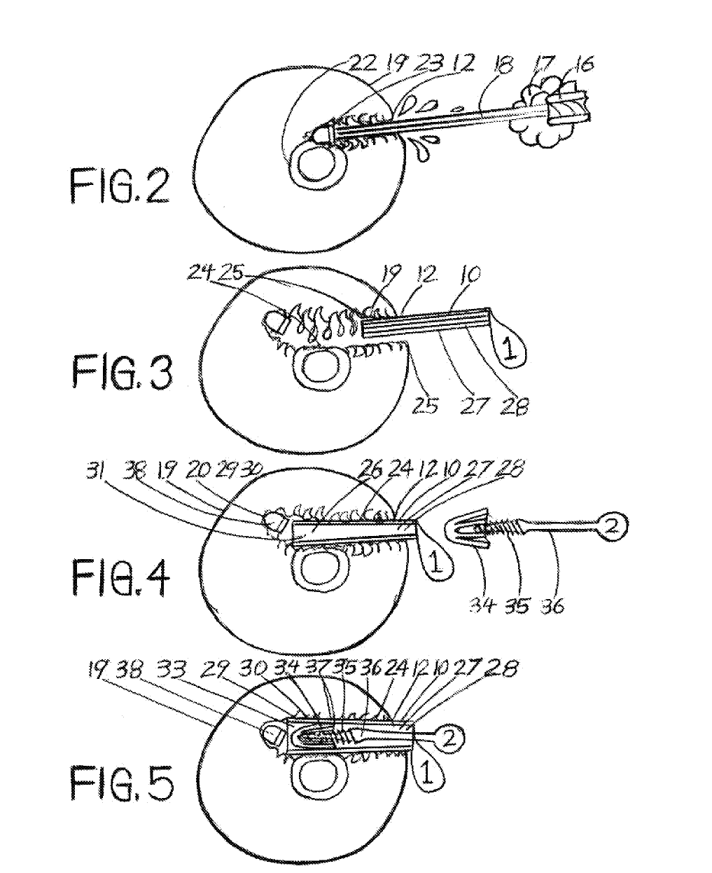

FIG. 2 illustrates a 16 typical gun barrel firing a 23 bullet with a 17 explosive propellant burst along an 18 bullet acceleration path into a 19 typical section of a body part at a 20 bullet impact point where the 21 bullet entering body part causes a 24 bleeding bullet wound hole that gets bigger as the bullet goes deeper into the body causing the 22 bullet impacting bone decelerating and coming to a stop in a fixed position as the 38 bullet at the end of the wound.

FIG. 3 shows an embodiment of the present invention 10 bullet plug device in a rolled configuration being inserted through the 12 bullet wound hole in the 19 typical section of a body part into the 24 bleeding bullet wound hole of the 11 typical bullet wound victim. The 15 bullet plug rolled spiral cylinder, which has a 27 coagulant coating for stopping bleeding and a 28 anesthetic coating for reducing pain contacts the 24 bleeding bullet wound hole and starts to have immediate first-aid healing effects at the 25 bullet plug entry point.

FIG. 4 shows an embodiment of the present invention 10 bullet plug device in the 12 bullet wound hole of the 19 typical section of a body part being unrolled by the drive mechanism actuated by the 13 bullet plug left handle and the 14 bullet plug right handle causing the enlarging of the 31 expanding unrolling spiral cone in diameter to fill the 24 bleeding bullet wound hole of the 11 typical bullet wound victim while the 27 coagulant coating and the 28 anesthetic coating on the 31 expanding unrolling spiral cone is causing the desired coagulant and anesthetic contact and pressure to shut off the bleeding and reduce the pain of the wound. The second part of the first aid system is the tunnel seal system. The second system includes the flexible 34 plunger seal that allows air to escape but not blood when the 35 plunger expansion screw is rotated by the 36 plunger stick causing the diameter of the 35 plunger to enlarge to fill the 37 wound seal area dose to the 38 bullet at the end of the wound to stop the bleeding and fill the bullet wound so that the victim does not immediately die because of loss of blood.

FIG. 5 shows an embodiment of the present invention 10 bullet plug device in a fully unrolled configuration with the 33 fully expanded spiral cone lodged inside the 32 compressed wound hole wall of the 11 typical bullet wound victim with the 29 bullet wound hole touching coagulant and the 30 bullet wound hole touching anesthetic providing a reduction in bleeding and a reduction in pain. The 24 bleeding bullet wound hole has been filled and pressure has been applied to all of the interior, cone shaped, surfaces of the 12 bullet wound hole with 27 coagulant coating and 28 anesthetic coating touching and compressed into the cone providing an instant blood shut off and a reduction of pain. The 38 bullet at the end of the wound can now be easily accessed for removal. After the bullet is removed, the 34 plunger on the end of the 36 plunger stick is inserted into the tunnel allowing air to escape, but not blood, until the 34 plunger reaches the bottom of the tunnel, where the 35 plunger expansion screw is rotated causing the diameter of the 34 plunger to enlarge and provide a 37 wound seal area. At this point, with bullet wound first aid applied, the bullet wound victim would seek out secondary medical attention for the bullet wound.

FIG. 6 shows the current invention 50 Open-O (Open Operation Device) 15 rolled spiral cylinder about to be inserted into a 42 typical incision, cut by a 40 typical scalpel. The 15 rolled spiral cylinder has a 43 expansion ratchet handle that serves as a mechanical drives to open and close the spiral. The 43 expansion ratchet handle may be equipped with a 44 light attached in order to illuminate the edges of the incision for ease of diagnosis and treatment.

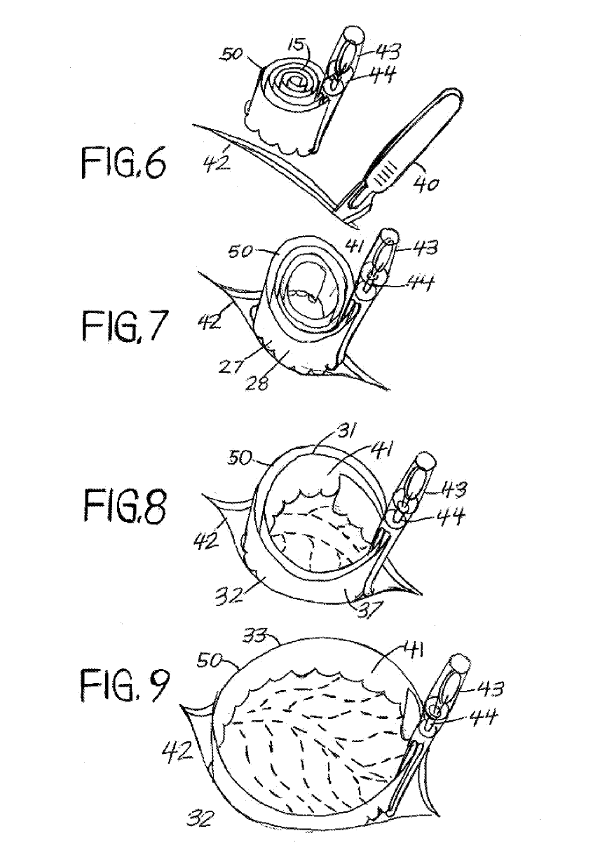

FIG. 7 shows the 15 rolled spiral cylinder inserted into the 42 typical incision with the 41 inner transparent walls of the spiral cone coming in contact with the 42 typical incision, which starts to apply he 27 coagulant coating in order to reduce the bleeding of the incision as well it starts to apply the 28 anesthetic coating in order to reduce the pain of the 42 typical incision.

FIG. 8 shows the 31 expanding unrolled spiral cone starting to enlarge the 42 typical incision as the 41 inner transparent walls of the spiral cone expand, driven by the 43 expansion ratchet handle and 44 light the 32 compressed wound hole wall, which illuminates the 37 wound seal area to give the surgeon an unprecedented amount of visual input.

FIG. 9 shows the 33 fully expanded spiral cone providing support to the 32 compressed wound hole wall in a 360 degree even pressured support that eliminates the points of pressure of previous art retractors. The 33 fully expanded spiral cone exposes the sub dermal organs of the body with in the circle of the 41 inner transparent walls of spiral cone, which are holding open the 42 typical incision in a circular, wide open manor, which allows a clean blood free open operation area that is illuminated by the 44 light. Once the operation is complete, the 50 Open-O Device is rewound into the 15 rolled spiral cylinder and removed leaving the edges of the incision with none of the bruises inherent with previous art retractors.

What is claimed is:

1. A first aid device for a gunshot bullet wound comprising a spiral wound cylinder of clear sheet material which is unrolled by a drive mechanism inside the bullet wound to create an expanded tunnel with which to fill the conical hole from a bullet wound to seal the bullet hole.

2. A first aid device for a gunshot bullet wound of claim 1 that has a clear, light illuminated, open tunnel to facilitate the removal of the bullet.

3. A first aid device for a gunshot bullet wound of claim 1 with a local anesthetic on the outside surface of the tunnel that reduces the pain of the wound.

4. A first aid device for a gunshot bullet wound of claim 1 with a blood coagulant on the outside surface of the tunnel to reduce the bleeding.

5. A first aid device for a gunshot bullet wound of claim 1 with a rolled up tunnel that opens up to expose and give easy access to the bullet at the end of the tunnel.

6. A first aid device for a gunshot bullet wound of claim 1 with a transparent tunnel that displays the lining of the bullet wound through the clear walls of the tunnel to the doctor during an exam or to the gunshot victim or to the person giving first aid.

7. A first aid device for a gunshot bullet wound of claim 1 that provides controlled opening and closing of the clear tunnel.

8. A first aid device for a gunshot bullet wound of claim 1 providing a ratchet mechanism that locks the tunnel size at any desired diameter.

9. A first aid device for a gunshot bullet wound of claim 1 with a vacuum fluid removal and suction function.

10. A first aid device for a gunshot bullet wound of claim 1 that conforms to the normal configuration of a bullet wound wherein the hole is small at the surface and expands in diameter as it enters the body using an interior diameter that grows automatically slightly larger than the outer diameter which naturally holds the truncated conical shaped tunnel inside the body at the opening of the wound.

11. A first aid device for a gunshot bullet wound of claim 1 is to provide an alternative to stuffing the bullet wound full of cotton, tampons or cloth to slow down the loss of blood until the gunshot victim can be rushed to a hospital for an emergency operation.

12. A first aid device for a gunshot bullet wound of claim 1 is to provide a balloon that expands inside the spiral wound cylinder to seal the inside of the wound and to seal the tunnel and stop the gunshot wound victim from bleeding to death.

13. A first aid device for a gunshot bullet wound of claim 1 that is individually wrapped in a sterilized convenient package.

14. A first aid device for a gunshot bullet wound of claim 1 that includes clear symbolic instructions printed on the sterile wrapper that provides any person with the knowledge to administer the bullet plug gunshot wound first aid device.

15. A first aid device for a gunshot bullet wound which slides inside the bullet wound and expands to fill the bullet wound.

16. The first aid device for a gunshot bullet wound of claim 15 including a coagulant coating to reduce the bleeding of the gunshot wound.

17. The first aid device for a gunshot bullet wound of claim 15 including an anesthetic coating to reduce the pain of the gunshot wound.

18. The first aid device for a gunshot bullet wound of claim 16 in which a balloon containing a CO2 cartridge is placed within the spiral wound cylinder so when the CO2 cartridge is activated the bullet plug is expanded to fill the bullet wound instantly.

19. A spiral wound cylinder of clear sheet material which is unrolled by a drive mechanism to create an expanded tunnel device that spreads open an incision to aid in a surgical operation by providing a clear, light illuminated, open tunnel to facilitate sub-dermal access.

20. A spiral wound cylinder of clear sheet material which is unrolled by a drive mechanism to create an expanded tunnel device that spreads open an incision to aid in a surgical operation of claim 19 which is aided by an optional local coagulant coating on the outside surface of the tunnel to reduce the bleeding of the incision, while an optional local anesthetic reduces the pain of the incision, while an optional anti-biotic or soap kills germs.
